(19) United States
(12) Patent Application Publication
(10) Pub. No.: US 2022/0363715 A1
(43) Pub. Date: Nov. 17, 2022

(54) Process for Purifying C1-INH

(71) Applicant: CSL Behring GmbH, Marburg (DE)

(72) Inventors: Roopsee Anand, Marburg (DE); Sabrina Huneke-Vogt, Lahntal (DE); Jennifer Krupka-Kloos, Marburg (DE); Martin Vey, Marburg (DE); Norbert Schulze, Hattersheim (DE)

(73) Assignee: CSL Behring GmbH, Marburg (DE)

(21) Appl. No.: 17/624,381

(22) PCT Filed: Jul. 3, 2020
(86) PCT No.: PCT/EP2020/068787

§ 371 Date: Jan. 3, 2022

(30) Foreign Application Priority Data
Jul. 4, 2019 (EP) 19184303.6

(51) Int. Cl. C07K 1/18; C07K 14/81; B01D 15/36; B01D 15/42

(52) U.S. Cl. CPC C07K 1/18; C07K 14/8121; B01D 15/363; B01D 15/426

(57) ABSTRACT

The present invention relates to a process for purifying C1-esterase inhibitor (C1-Inh), and more in particular a Cl-Inh concentrate.

The present invention relates to a process for purifying C1-esterase inhibitor (C1-INH), and more in particular a C1-INH concentrate.

C1-INH, a protein of the pathway of complement activation, is an inhibitor of proteases present in the plasma which controls C1-activation by forming covalent complexes with activated C1r and C1s. It also “controls” important blood coagulation enzymes, such as plasma prekallikrein, factors XI and XII, but also plasmin.

C1-INH deficiency is for instance associated with hereditary angioedema (HAE) caused by lack of C1-INH (HAE type I) or a reduced activity of C1-INH (HAE type II). C1-INH deficiency may also be caused by consumption of C1-INH due to neutralisation of enzymes generated when blood comes into contact with surfaces such as in a heart-lung machine, but also in disease courses initiating the coagulation cascade, such as immune complexes appearing in the context of chronic, in particular rheumatic disorders. Currently, C1-INH protein replacement must be considered as the gold standard in the prevention or treatment of acute HAE. This holds particularly true for commercially available human blood plasma derived C1-INH, which reportedly has a more natural functionality than a commercially available recombinant C1-INH produced in transgenic rabbits which is not identical to the human C1-INH protein (Feussner et al., Transfusion 2014 October; 54(10):2566-73, doi: 10.1111/trf.12678). Further therapeutic applications that have been considered include the use of C1-INH in prevention, reduction and/or treatment of ischemia reperfusion injury (cf. WO 2007/073186).

Isolation and/or purification of C1-INH from human blood plasma is a known but more or less expensive and in particular most often a very time consuming process. The different methods proposed for producing C1-INH from blood plasma include various separation methods such as affinity chromatography, ion exchange chromatography, gel filtration, precipitation, and hydrophobic interaction chromatography. Using any of these methods alone is generally insufficient to purify C1-INH, and in particular C1-INH concentrates, sufficiently, hence various combinations thereof have been proposed in the prior art.

EP 0 698 616 B describes the use of anion exchange chromatography followed by cation exchange chromatography. EP 0 101 935 B describes a combination of precipitation steps and hydrophobic interaction chromatography in a negative mode to arrive at a 90% pure C1-INH preparation at a yield of about 20%. U.S. Pat. No. 5,030,578 describes PEG precipitation and chromatography over jacalin-agarose and hydrophobic interaction chromatography in a negative mode.

WO 01/46219 describes a process wherein a C1-INH-containing starting material is treated twice with an anion exchanger under acidic conditions (the pH is respectively set to pH 5.5). The first ion exchange step is followed by PEG precipitation and then the second ion exchange step, like in the manufacture of Cinryze®, which is known to still comprise ACT (cf. Feussner et al., Transfusion 2014 October;54(10):2566-73, doi: 10.1111/trf.12678).

Out of four commercially available C1-INH concentrates for treatment of angioedema, three are plasma derived. The latter are sold under the tradenames Berinert®, Cinryze® and Cetor®. These C1-INH concentrates are prepared according to different proprietary processes (cf. in Feussner et al., Transfusion 2014 October;54(10):2566-73, doi: 10.1111/trf.12678). These proprietary processes are known to respectively involve a sequence of steps including, but not limited to: cryoprecipitation, ion-exchange chromatography, precipitation, pasteurization, ultra-and/or diafiltration, and/or hydrophobic interaction chromatography. These multi-step processes are well established, robust, and reliable. They yield bulk products with just tiny amounts of accompanying proteins detectable therein, in particular alpha-1-antichymotrypsin (ACT).

Despite a molecular weight of only half that of C1-INH, ACT co-purifies during manufacturing of plasma derived C1-INH preparations such as the aforementioned Berinert®, but also HAEGARDA®, Cinryze® or Cetor®. Among the aforementioned, Berinert® (and HAEGARDA®) have the lowest ACT-levels (Feussner et al., Transfusion 2014 October;54(10):2566-73, doi: 10.1111/trf.12678), namely of well below 5 μg/IU C1-INH.

Self-evidently, manufacturers strive to provide a product which is as pure as possible. Tiny amounts of accompanying proteins are accepted so long as they have no negative impact on effect and safety of the actual end product. But in principle even tiny amounts of accompanying proteins are unwanted.

It may be possible to isolate 100% pure C1-INH from a corresponding preparation on a laboratory scale. Yet achieving such a high degree of purity on an industrial scale is not easily and reliably feasible for economic reasons, and be it only due to loss of product associated with time-consuming processes. The raw material from which such preparations are derived, i.e. blood, is in other words not abundant enough to allow for excessive losses of active material only to achieve an extremely high degree of purity.

Irrespective thereof manufacturers constantly strive to improve their respective processes. Corresponding efforts at improving established multi-step processes like the one used in the manufacture of Berinert® may mean that individual steps are reviewed and amended. Such changes may for instance lead to a higher yield, a faster processing time, etc. Or small changes may simply become necessary for other reasons—such as changes in the supply chain (e.g. availability of third party materials used in a process, etc.). Every small change may possibly come with inconveniences. For instance amounts of accompanying proteins, although remaining very tiny, may happen to be less tiny than before a corresponding change.

The presence of tiny amounts of ACT in C1-INH preparations has long been—and still is—considered to cause no harm. Yet it is still desirable to provide C1-INH preparation which are as pure as possible and therefore to further reduce the ACT-content in C1-INH preparations that have been obtained from blood plasma via multi-step processes.

The ACT protein is comprised of 423 amino acids including a 25 residue signal peptide at the amino terminus which is cleaved from the mature protein. The total molecular weight of ACT is approximately 55 to 66 kDa due to heavy glycosylation at multiple sites. It has a typical serpin structure (Baker et al., SERPINA3 (aka alpha-1-antichymotrypsin), Front. Biosci. 2007 (12), 2821-2835). ACT finds cognate proteases to form a serpin: protease complex, which is cleared from the circulating plasma by the liver at a rate 10 to 50 times more rapidly than ACT alone (Mast et al., Biochem. 1991 (30), 1723-1730). ACT is an acute phase protein, with plasma levels increasing in response to inflammation. Its role in acute phase response is to act as an inhibitor of several serine proteases, most pronouncedly leukocyte cathepsin G (Crispe, J. Immunol. 2016 (196), 17-21). Cathepsin G is released at the site of inflammation, where it kills and degrades pathogens, remodels tissues and activates pro-inflammatory cytokines and receptors. Excessive or prolonged activity of cathepsin G and resulting tissue damage is averted by serpin regulation, e.g. ACT. The concentration of ACT in human plasma normally ranges around 400 mg/l (Hollander et al., BMC Pulm. Med. 2007 (29), 7).

Commercially available ACT preparations are known to comprise ACT in concentrations ranging from 31 μg/IU C1-INH in the case of Cinryze® or below 5 μg/IU C1-INH in the case of Berinert® or HAEGARDA®.

To ensure levels of ACT below 5 μg/IU C1-INH or even lower there is a need for means enabling further purification, or “polishing”, of C1-INH preparations obtained from blood plasma by means of multi-step processes with minimal loss of C1-INH product. Human blood plasma is generally hard to come by in sufficient amounts to satisfy existing needs. C1-INH preparations obtained in established and optimized multi-step processes are highly concentrated. Further purification or polishing of existing C1-INH preparations obtained from blood plasma therefore should not entail inconveniences such as loss of product, unnecessary dilution, or the like.

To solve this problem, the present invention provides a process for the depletion of 1-antichymotrpysin (ACT) from a C1-INH preparation obtained from blood plasma by means of a preceding process involving several steps, wherein the depletion of ACT from the C1-INH preparation is carried out by anionic exchange chromatography.

The ACT-concentration in the C1-INH preparation constituting the starting material may be e.g. below 100, 50, 35, 30, 25, 20, 15, preferably below 35, more preferably below 10, most preferably below 5 μg/IU C1-INH.

Inventors were able to achieve substantial reduction of the ACT-content still present in C1-INH preparations essentially without accompanying loss of C1-INH product or unnecessary dilution, providing an efficient polishing step enhancing the purity and safety of existing C1-INH preparations that are intended to be used as a medicament.

Preferably the C1-INH preparation is one obtained from blood plasma by means of a preceding process involving several steps including, but not limited to: cryoprecipitation, ion-exchange chromatography, precipitation, pasteurization, ultra- and/or diafiltration, and/or hydrophobic interaction chromatography.

Corresponding C1-INH preparations are known under the tradenames Berinert®, Haegarda®, Cinryze® and Cetor®. The manufacturing processes used to produce them yield preparations with already very low ACT contents.

It is noted in the context that the manufacture of Cinryze® and/or Cetor® reportedly involves cryoprecipitation, various ion-exchange chromatography steps, PEG precipitation, pasteurization, filtration, and lyophilisation, whereas the manufacture of Berinert® reportedly involves cryoprecipitation, ion-exchange chromatography, quaternary aminoethyl adsorption, ammonium sulphate precipitations, pasteurization, hydrophobic interaction chromatography, filtration and lyophilisation. As the latter yields even purer product than the former, i.e. with lower ACT contents, further depletion of ACT from the latter may yield an even better C1-INH preparation obtained from blood plasma than already obtained in the case of Berinert®-manufacturing process with even less resources and is therefore particularly preferred.

Hence it is particularly preferred that the C1-INH preparation is one obtained from blood plasma by means of a preceding process involving several steps including, but not limited to hydrophobic interaction chromatography.

Preferably the process according to the invention comprises the following steps:
(i) loading an anionic exchange chromatography column comprising a stationary phase with the C1-INH preparation under first conditions under which C1-INH and ACT bind to the stationary phase;
(ii) an optional step of washing the charged column;
(iii) application of second conditions so as to elute ACT by means of a mobile phase;
(iv) application of third conditions so as to elute C1-INH by means of a mobile phase.

Preferably the second condition in aforementioned step (iii) consists in the use of an elution buffer of an ionic strength A and the third condition in aforementioned step (iv) consists in the use of an elution buffer of an ionic strength B, wherein ionic strengths A and B are different.

The transition from ionic strength A to ionic strength B is preferably achieved by means of a salt concentration gradient, or by means of a step elution using elution buffers EB_{A} and EB_{B} with different salt concentrations c_{A} and c_{B}.

Preferably elution buffer EB_{A} consists of a buffer having a conductivity of 18.7 to 20.20 mS/cm at 25° C., preferably of 18.9 to 19.8 mS/cm at 25° C., most preferably of 19.2 mS/cm at 25° C., or more preferably 10 mM Tris, 175-190 mM NaCl, preferably 175-185 mM NaCl, most preferably 180 mM NaCl, pH 7.2

While elution buffer EB_{A} is chosen such that C1-IHN remains bound to the charged column, wherein a least one or more contaminating proteins do not bind to the charged column, the elution buffer EB_{B} is chosen such that essentially all C1-INH bound to the charged column is no longer bound and can be collected in the eluate.

By the precise selection of elution buffer EB_{A} and EB_{B} the removal of contaminating proteins like ACT becomes possible without loss or with only minimal loss of C1-INH.

The elution buffer EB_{B} has a conductivity higher than 25.3 mS/cm at 25° C., or higher than 30.4 mS/cm at 25° C., or higher than 38.5 mS/cm at 25° C., or higher than 47.7 mS/cm at 25° C., or higher than 54.8 mS/cm at 25° C., or higher than 63.4 mS/cm at 25° C., or higher than 69.7 mS/cm at 25° C., or higher than 77.8 mS/cm at 25° C., or equal or higher than 84.4 mS/cm at 25° C., wherein elution buffer EB_{B} is eluting essentially all C1-INH still bound to the anionic exchange chromatography. The skilled person can easily determine the necessary conditions.

More preferably the elution buffer EB_{B} consists of 10 mM Tris, 250 mM NaCl, pH 7.2; or 10 mM Tris, 300 mM NaCl, pH 7.2; or 10 mM Tris, 400 mM NaCl, pH 7.2; or 10 mM Tris, 500 mM NaCl, pH 7.2; or 10 mM Tris, 600 mM NaCl, pH 7.2; or 10 mM Tris, 700 mM NaCl, pH 7.2; or 10 mM Tris, 800 mM NaCl, pH 7.2; or 10 mM Tris, 900 mM NaCl, pH 7.2, or 10 mM Tris, 1M NaCl, pH 7.2.

| Buffer Composition | Measured conductivity @ 25 (±0.5) ° C. |
|---|---|
| 10 mM Tris, 250 mM NaCl pH 7.2 | 25.3 mS/cm |
| 10 mM Tris, 300 mM NaCl pH 7.2 | 30.4 mS/cm |
| 10 mM Tris, 400 mM NaCl pH 7.2 | 38.5 mS/cm |
| 10 mM Tris, 500 mM NaCl pH 7.2 | 47.7 mS/cm |
| 10 mM Tris, 600 mM NaCl pH 7.2 | 54.8 mS/cm |
| 10 mM Tris, 700 mM NaCl pH 7.2 | 63.4 mS/cm |
| 10 mM Tris, 800 mM NaCl pH 7.2 | 69.7 mS/cm |
| 10 mM Tris, 900 mM NaCl pH 7.2 | 77.8 mS/cm |
| 10 mM Tris, 1000 mM NaCl pH 7.2 | 84.4 mS/cm |

A process of the invention does combine buffers EB_{A} and EB_{B} as described below:

a)
(i) elution buffer EB_{A} has a conductivity of 18.7 to 20.2 mS/cm at 25° C., preferably of 18.9 to 19.8 mS/cm at 25° C., most preferably of 19.2 mS/cm at 25° C.
(ii) elution buffer EB_{B} has a conductivity being higher than 25.3 mS/cm at 25° C. or higher than 30.4 mS/cm at 25° C., or higher than 38.5 mS/cm at 25° C., or higher than 47.7 mS/cm at 25° C., or higher than 54.8 mS/cm at 25° C., or higher than 63.4 mS/cm at 25° C., or higher than 69.7 mS/cm at 25° C., or higher than 77.8 mS/cm at 25° C., or equal or higher than 84.4 mS/cm at 25° C., wherein elution buffer EB_{B} is eluting essentially all C1-INH still bound to the anionic exchange chromatography column after elution step (i).

or

b)
(i) elution buffer EB_{A} consists of 10 mM Tris, 175-190 mM NaCl, preferably 175-185 mM NaCl, most preferably 180 mM NaCl, pH 7.2, and
(ii) elution buffer EB_{B} has a conductivity being higher than 25.3 mS/cm at 25° C. or higher than 30.4 mS/cm at 25° C., or higher than 38.5 mS/cm at 25° C., or higher than 47.7 mS/cm at 25° C., or higher than 54.8 mS/cm at 25° C., or higher than 63.4 mS/cm at 25° C., or higher than 69.7 mS/cm at 25° C., or higher than 77.8 mS/cm at 25° C., or equal or higher than 84.4 mS/cm at 25° C., wherein elution buffer EB_{B} is eluting essentially all C1-INH still bound to the anionic exchange chromatography column after elution step (i).

or

c)
(i) elution buffer EB_{A} consists of 10 mM Tris, 175-190 mM NaCl, preferably 175-185 mM NaCl, most preferably 180 mM NaCl, pH 7.2, and
(ii) elution buffer EB_{B} consists of 10 mM Tris, 250 mM NaCl, pH 7.2 or 10 mM Tris, 300 mM NaCl, pH 7.2; or 10 mM Tris, 400 mM NaCl, pH 7.2; or 10 mM Tris, 500 mM NaCl, pH 7.2; or 10 mM Tris, 600 mM NaCl, pH 7.2; or 10 mM Tris, 700 mM NaCl, pH 7.2; or 10 mM Tris, 800 mM NaCl, pH 7.2; or 10 mM Tris, 900 mM NaCl, pH 7.2, or 10 mM Tris, 1M NaCl, pH 7.2.

The inventors found that such conditions are particularly useful in that they allow for a depletion of ACT without essential loss of C1-INH, i.e. the C1-INH recovery is higher than 90%, preferably at least 95% under such circumstances, while ACT is decreased substantially, i.e. by 50% or more.

The stationary phase material used in the anion exchange chromatography belongs to the type either of weak anion exchangers, such as Capto® DEAE (sold by GE, using diaminoethyl as a functional group) or—preferably—of strong anion exchangers, such as Q HP resin, Capto® Q Impres resin, Capto® Q resin (all sold by GE, all with quaternary ammonium as a functional group) or Fractogel® TMAE, Eshmuno® H (sold by Merck, with trimethylamonethyl as a functional group).

Among the aforementioned, resins with ordinary ammonium as a functional group are the most preferred.

It is preferred that the C1-INH preparation is derived from human blood plasma.

It is understood that blood plasma is derived from blood wherein blood means a body fluid found in humans and other animals. This means that the process according to the invention may serve to polish C1-INH preparations derived from all kinds of animal blood plasma, yet preferably human blood plasma, wherein C1-INH preparations obtained from human blood plasma are particularly preferred due to their importance in the treatment of e.g. haemophilia in humans suffering therefrom.

It is furthermore preferred that the C1-INH preparation consists essentially of C1-INH and ACT dissolved in a medium.

The invention aims at further polishing preferably such preparations, irrespective of the way in which they have been obtained. Preferably, the ACT content is below 100, 50, 35, 30, 25, 20, 15, preferably below 35, more preferably below 10 μg ACT/IU C1-INH and most preferably below 5 μg ACT/IU C1-INH.

The invention further provides a C1-INH preparation derived from blood plasma that can be obtained by using a process according to any one of methods described above.

While C1-INH preparations obtained from blood plasma are in principle known, corresponding preparations from which ACT has been depleted as presently described are not known from the prior art. While it may in principle be possible to obtain highly purified C1-INH, i.e. without any subsisting trace of ACT therein, the process according to the present invention very substantially reduces the ACT level, but not so as to exclude ACT completely.

In the following, the present invention will be described in more details by means of figures and examples, wherein the figures depict the following:

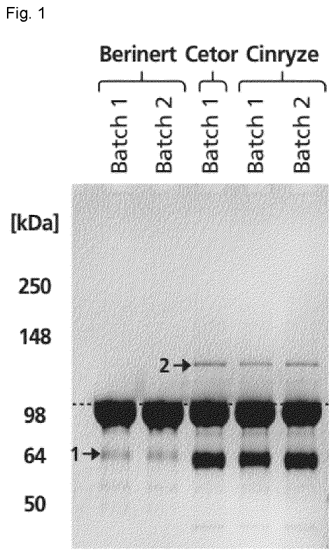

FIG. 1: electrophoresis gels showing the presence of ACT in commercially available C1-INH preparations according to the prior art;

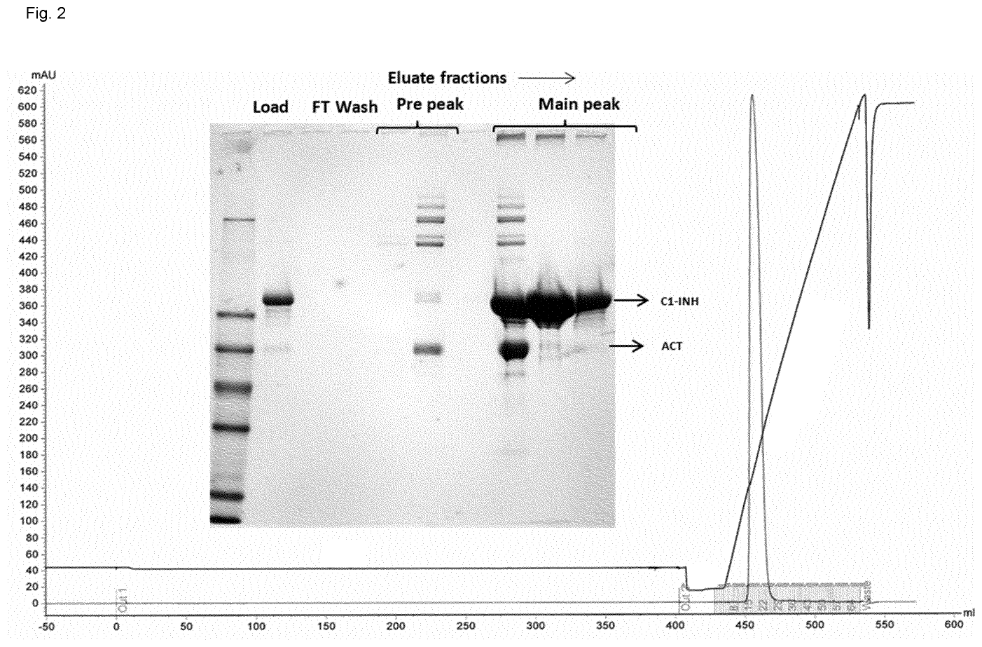

FIG. 2: a chromatogram of an AEX carried out in a bind/elute mode on a C1-INH preparation obtained by an established industrial process and SDS-page gel of eluate samples obtained in the same experiment;

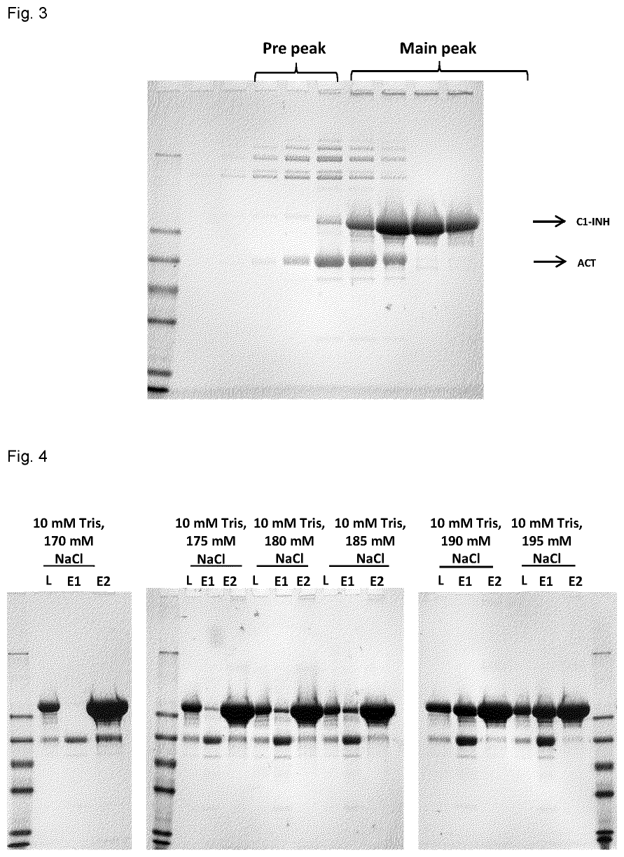

FIG. 3: an SDS-page gel of eluate samples obtained using AEX carried out in a bind/elute mode on a C1-INH preparation obtained by an established industrial process;

FIG. 4: SDS-page gels of eluate samples obtained from AEX carried out in a bind/elute mode on a C1-INH preparation obtained by an established industrial process comparing various elution buffers;

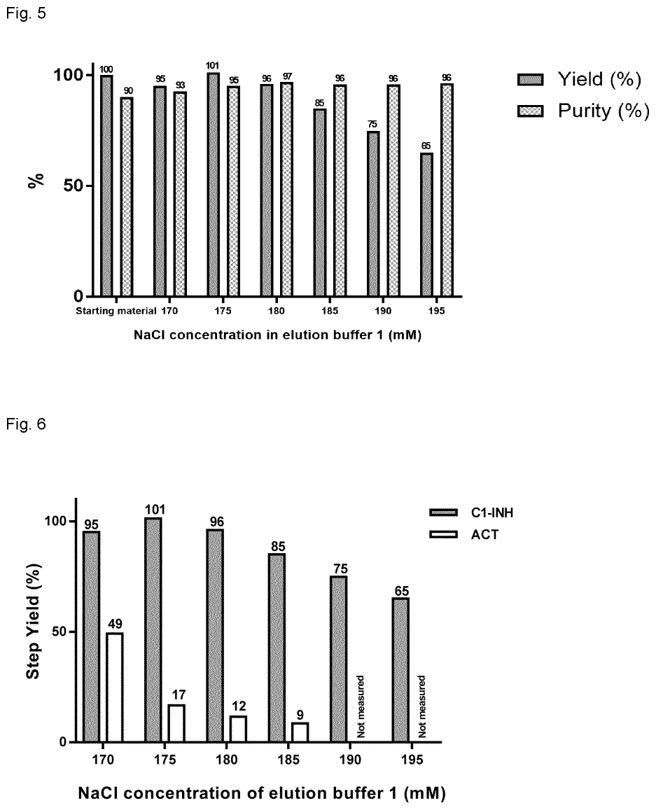

FIG. 5: a diagram summarising purity and extent of C1-INH recovery in a second AEX chromatography eluate using a buffer of high ionic strength C1-INH depending on the salt content of eluent buffer used in a a first elution from the same AEX matrix using a low ionic strength buffer at NaCl concentrations from 170 to 195 mM, corresponding to buffers having a conductivity from 18.7 to 20.7 mS/cm at 25° C.

FIG. 6: a diagram depicting the amount of C1-INH and ACT in a second AEX chromatography eluate using a buffer of high ionic strength depending on the salt content of eluent buffer used in a a first elution from the same AEX-matrix using a low ionic strength buffer at NaCl concentrations from 170 to 195 mM, corresponding to buffers having a conductivity from 18.68 to 20.7 mS/cm at 25° C.

In the context of the present invention, the following definitions apply:

In the claims and in the description of the invention “C1-INH”, C1-INH″, “C1-INH preparation” and “C1-INH preparation” are concurrently used to designate concentrates containing the protein C1-esterase inhibitor and in particular liquid concentrates containing the protein C1-esterase inhibitor. When referring to the technical background and/or prior art, “C1-INH” may also mean the protein as such, e.g. in the context of discussing C1-INH deficiency.

Throughout the present application/patent
“HIC” means hydrophobic interaction chromatography;
“AEX” means anion exchange chromatography;
“AEX resin” means a resin used as stationary phase in AEX;
“strong AEX resin” means a highly ionized AEX resin that can be used over a broad pH range;
“weak AEX resin” means a resin of which the degree of ionization strongly depends on pH;
“BC” means binding capacity of a chromatography column;
“negative mode” or “flow through mode”, or “flow through” designates a way of carrying out a chromatography under conditions under which a target compound (e.g. C1-INH) does not bind to the stationary phase of a chromatography column;
“binding mode”, “binding and elution” or “positive mode” stands for a chromatography first carried out under conditions under which a target compound (e.g. C1-INH) binds to the stationary phase of a chromatography column and then under conditions under which the same compound is eluted from the chromatography column;
when a “compound binds to the stationary phase”, this is intended to mean is adsorbed by or retained on the stationary phase without the structural integrity of the compound being affected, preferably not by covalent bonds or chemisorption, but rather by physisorption;
“WFI” means “water for injection”;
“concentration gradient” designates the gradual variation of the concentration of a dissolved substance in a solution from a higher concentration to a lower concentration,
“step elution” means a sudden transition from the first to the second concentration instead of a continuous transition as in a concentration gradient, wherein the concentration is gradually lowered;
“%” means “% by weight” unless otherwise stated;
“precipitant” is an agent triggering precipitation of proteins;
“eluate fraction” designates a fraction of the mobile phase stream emerging from a chromatographic column irrespective of whether specific analytes comprised therein were previously bound to or retained by the stationary phase (as in a positive mode as mentioned herein) or not (as in a negative mode as mentioned herein).

In the following, the present invention will be explained in more detail by making reference to the figures.

FIG. 1 shows an electrophoresis gel of commercially available C1-INH preparations known from prior art. The dotted line indicates the molecular weight of C1-INH (105 kD). The differences between commercially available C1-INH preparations derived from blood plasma known under the tradenames Berinert®, Cetor® and Cinryze® can clearly be distinguished. Lanes 1 to 5 in this gel show that Berinert®, Cetor® and Cinryze® comprise traces of ACT, wherein Berinert® contains the smallest amount thereof (FIG. 1, 1), as discussed in more detail by Feussner et al. Berinert®, Cetor® and Cinryze®, but also Haegarda® are, respectively, C1-INH preparations obtained from blood plasma by means of a process involving several steps. The steps involved in the manufacture of Berinert®, Cetor® and Cinryze® have been described previously (Feussner et al., Transfusion 2014 October;54(10):2566-73, doi: 10.1111/trf.12678).

FIG. 2 represents a chromatogram of an AEX chromatography experiment according to the invention and an SDS-page gel analysing elute samples from that experiment; column load was a C1-INH concentrate taken from the production of C1-INH preparation Berinert®, i.e. the eluate of the last hydrophobic interaction chromatography step in the preparation of Berinert® (diluted 1:25); binding capacity BC was 15 mg protein/mL resin; separation was carried out by means of a salt gradient from 30 mM to 1000 mM NaCl. The pre-peak eluate sample clearly comprises ACT as can be seen on the SDS-page gel in FIG. 2 (cf. lane 6). Thus, the chromatogram and SDS-page gel of FIG. 2 demonstrate the depletion of ACT from a C1-INH preparation obtained by a process involving multiple steps and comprising already a very low concentration of ACT by using AEX chromatography.

FIG. 3 represents an SDS-page gel of eluate samples obtained from an AEX chromatography experiment with the same column load and binding capacity as in the experiment represented in FIG. 2, but using a less steep salt gradient, i.e. 30 mM to 515 mM NaCl. The SDS-page gel of FIG. 3 demonstrates the depletion of ACT from a C1-INH preparation by using AEX chromatography.

FIG. 4 represents SDS-page gels of eluate samples obtained from AEX chromatography experiments using step elution with varying salt concentrations of the first elution buffer destined to elute ACT from the stationary phase. “E1” designates a lane corresponding to a sample of the respective eluate 1, “E2” designates a lane corresponding to a sample of the respective eluate 2. It can clearly be seen that the extent of depletion of ACT from C1-INH preparations increases with increasing salt concentration, whereas the extent of C1-INH recovery in eluate 2 decreases with increasing salt concentration.

FIG. 5 shows the purity and extent of C1-INH recovery in eluate 2 depending on the salt content of eluent buffer 1 for different eluate buffers 1 in comparison. As can be inferred from FIG. 5, eluent buffer 1 with NaCl concentration in the range of 175-190, preferably 175-185, most preferable 180 mM NaCl is best suited for maximum depletion of ACT without essential loss of C1-INH from C1-INH preparations.

FIG. 6 shows the amount of C1-INH and ACT in in eluate 2 depending on the salt content of eluent buffer 1 for different eluate buffers 1 in comparison. FIG. 6 shows that at 175 mM NaCl or above in the buffer used for the first elution a much better depletion of ACT is achieved in the final product compared to a lower ionic strength in eluent buffer 1 and that at an ionic strength of above 190 mM of eluent buffer 1 the yield of C1-INH becomes too low to be commercially viable.

C1-INH preparations derived from animal blood, and in particular human blood, are nowadays obtained by various multi-step processes. The established processes departing from human blood plasma include steps of cryoprecipitation, ion-exchange chromatography, quaternary aminoethyl adsorption, ammonium sulphate precipitations, pasteurization and hydrophobic interaction chromatography (process steps included in the manufacture of Berinert®, see Feussner et al., or EP 0 101 935) or cryoprecipitation, various steps of ion-exchange chromatography, precipitation with PEG (in particular PEG-4000) and pasteurization (process steps included in the manufacture of Cinryze®/Cetor®). These processes have in common that they comprise precipitation steps. Additional last steps are filtration and lyophilisation. The present invention proposes to add the additional step of anion exchange chromatography to further purify C1-INH preparations obtained by multi-step processes like the aforementioned ones, thus providing a “polishing” step to enhance the safety of existing products even further. That polishing step may take place before filtration and lyophilisation, but otherwise it is the last step following a sequence of other steps yielding a C1-INH concentrate or C1-INH preparation which nearly corresponds to the final product.

Surprisingly, the use of AEX chromatography in an additional polishing step enables still further depletion of ACT from C1-INH preparations without essential loss of C1-INH and without unnecessary dilution. Although AEX chromatography has been known for a long time, and even though it's use in the preparation of C1-INH concentrates has occasionally been mentioned, also in the context of separating C1-INH from accompanying proteins, it has so far not been used to the specific aim of depleting ACT from C1-INH preparations obtained by multiple step processes, wherein ACT still subsists as an impurity despite considerable efforts having been made in the past to obtain essentially pure C1-INH preparations.

That this is possible at all could not have been expected in view of this background. Quite surprisingly, otherwise well-established processes enabling production of C1-INH preparations on an industrial scale may still be improved via the inclusion of a corresponding polishing step. i.e. at a comparably late stage of the respective process.

In the following, the invention will be described in more detail by referring examples.

### Examples

### Example 1

A C1-INH concentrate taken from the production of C1-INH preparation Berinert®, i.e. the eluate of the last hydrophobic interaction chromatography (HIC) step in the preparation of Berinert® was diluted 1:25 to decrease the concentration of the chaotropic agent ammonium sulphate (AS) employed in the preceding HIC so as to enable protein binding. An AEX chromatography was then carried out in bind/elute mode, i.e. the starting material was loaded onto a column using a binding buffer, subsequently washed with a wash buffer, and lastly eluted by applying a salt gradient. Composition of buffers and gradient and further details are disclosed in Table a-1, the corresponding chromatogram and SDS page gel are shown in FIG. 2.

TABLE a-1

| | |
|---|---|
| Binding buffer | 10 mM Tris, 32 mM AS, pH 7.2 |
| BC | 15 mg protein/mL resin |
| Wash buffer | 10 mM Tris, 30 mM NaCl, pH 7.2 |
| Elution gradient | from 10 mM Tris, 30 mM NaCl, pH 7.2, to 10 mM Tris, 1M NaCl, pH 7.2 |

Example 1 thus demonstrates the depletion of ACT from a C1-INH preparation obtained by a process involving multiple steps and comprising already a very low concentration of ACT by using AEX chromatography.

### Example 2

An AEX chromatography was carried out as described in example 1, yet with an elution gradient from 10 mM NaCl to 515 mM NaCl. The corresponding chromatogram is shown in FIG. 3 discussed herein above. Example 2 thus demonstrates the depletion of ACT from a C1-INH preparation obtained by a process involving multiple steps and comprising already a very low concentration of ACT by using AEX chromatography.

Example 3

### Starting Material

Product obtained in the Berinert process, a highly purified C1-INH concentrate obtained by hydrophobic interaction chromatography (HIC) similar to the one described by Feussner et al. (doi: 10.1111/trf.12678) (batch No. 20181219-HW) stored at −20° C. was used. This material was dialyzed against 10 mM Tris, 32 mM AS pH 7.2 overnight at 4° C. and then subjected to anion exchange chromatography experiments comparing elution buffers 1 with different salt concentrations as represented in the following table b-1.

TABLE b-1
Experiments using different buffer 1

| | | | conductivity | |
|---|---|---|---|---|
| exper- | buffer/ | | (25(±0.5)° C. | column |
| iment | solution | composition | mS/cm | load |
| 1-6 | equilibration | 10 mM Tris, 32 mM | | |
| | buffer | AS pH 7.2 | | |
| 1-6 | wash buffer | 10 mM Tris, 30 mM NaCl pH 7.2 | | |
| 1 | elution buffer 1 | 10 mM Tris, 170 mM NaCl pH 7.2 | 18.7 | c |
| 2 | | 10 mM Tris, 175 mM NaCl pH 7.2 | 18.9 | c |
| 3 | | 10 mM Tris, 180 mM NaCl pH 7.2 | 19.2 | b |
| 4 | | 10 mM Tris, 185 mM NaCl pH 7.2 | 19.8 | b |
| 5 | | 10 mM Tris, 190 mM NaCl pH 7.2 | 20.2 | c |
| 6 | | 10 mM Tris, 195 mM NaCl pH 7.2 | 20.7 | a |
| 1-6 | elution buffer 2 | 10 mM Tris, 1M NaCl pH 7.2 | | |
| 1-6 | regeneration | 2M NaCl | | |
| | solution | | | |

### Substances and Equipment

### Substances:

De-ionised Water obtained via Milli-Q.

### Equipment:

| | |
|---|---|
| Chromatography Device | ÄKTA avant 25 |
| Software | Unicorn 6.4 |
| pH meter | Knick |
| conductivity meter | Knick |
| weighing balance | Sartorius |
| magnetic stirrer | Thermo Scientific |
| column | GE Healthcare, resin: Q HP, Lot No. 10270237 |
| column dimensions | inner diameter 0.77 cm, bed height 10 cm, column |
| | volume 4.7 mL |

TABLE b-2
column loads

| | a | | b | | c | |
|---|---|---|---|---|---|---|
| pH | 7.18 | | 7.16 | | 7.15 | |
| conductivity | 9.18 | mS/cm | 8.95 | | 8.97 | |
| | (22.1° C.) | | (21.7° C.) | | (22.9° C.) | |
| OD¹ | 0.1026 | | 0.0885 | | 0.0911 | |
| | (0.283 mg/mL) | | (0.244 mg/mL) | | (0.251 mg/mL) | |
| Volume² | 205.6 | mL | 241 | mL | 234 | mL |
| total protein³ | 58.1 | mg | 58.8 | mg | 58.7 | mg |
| BC⁴ | 12.4 | mg/mL | 12.5 | mg/mL | 12.5 | mg/mL |

¹1 OD = 2.76 mg/mL protein;
²volume = volume used for loading onto column, also termed “column load”
³total protein = total protein amount loaded
${}^{4} BC = Binding Capacity = \frac{total protein ( mg )}{column volume ( mL )}$

TABLE b-3
ÄKTA program

| | |
|---|---|
| Pump A1 and Buffer | equilibration buffer |
| Pump A2 | wash buffer |
| Pump A3, A4, A6 | different elution buffer 1 |
| Pump B1 | elution buffer 2 |
| Pump A5 | 2M NaCl |
| Pump S1 | column load |

TABLE b-4
Example of ÄKTA program

| Step | Volume | Inlet | Flow rate (cm/h; mL/min) | Outlet |
|---|---|---|---|---|
| Equilibration | 5 CV | A1 | 150; 1.2 | Waste |
| Sample Application | 241 mL | S1 | 130; 1.0 | Waste |
| Washing | 5 CV | A2 | 130; 1.0 | Waste |
| Elution 1 | 5 CV | A3 | 150; 1.2 | Outlet 2 |
| Elution 2 | 5 CV | B1 | 150; 1.2 | Outlet 3 |
| Regeneration | 5 CV | A5 | 130; 1.0 | Waste |

Calculation of yields is based on volume of the respective column load a-c (see table b-1 above) and on the volumes of eluate 1 and 2 (23.5 mL, respectively).

### Analytics

Samples from “column load” (L), “eluate 1” (E1) and “eluate 2” (E2) were analyzed by SDS-PAGE. Corresponding SDS-PAGE gels are represented in FIG. 4. Samples from “Column load” and “Eluate 2” were additionally tested for C1-INH activity in quality control laboratories.

Results of the comparison are summarised in the graph represented in FIG. 5, showing yields of C1-INH recovered in the respective eluate 2 in comparison to purity in %. In accordance therewith, eluent buffer 1 with NaCl concentration in the range of 175-190 mM NaCl, preferably 175-185 mM NaCl, most preferably 180 mM NaCl is best suited for maximum depletion of ACT without essential loss of C1-INH from C1-INH preparations. This corresponds to conductivities of eluent buffer 1 from 18.7 to 20.2 mS/cm at 25° C., preferably from 18.9 to 19.8 mS/cm at 25° C., most preferably of 19.2 mS/cm at 25° C. Eluent buffer 2 needs to be high enough to elute all C1-INH still bound to the anionic exchange chromatography columns and needs to be higher than 21.6. mS/cm at 25° C. One example for an eluent buffer 2 is a buffer composition of 10 mM Tris, 1M NaCl, pH 7.2

FIG. 6 shows that using an eluent buffer 1 of 170 mM NaCL (18.7 mS/cm at 25° C.) there is already some reduction of ACT but still 49% of the starting amount of ACT in the final product whereas using an eluent buffer 1 of 175 mM NaCl (18.9 mS/cm at 25° C.) there is only 17% of the starting amount of ACT in the final product. This illustrates how important the fine tuning of the ionic strength/conductivity of the eluent buffer 1 needs to be to allow a maximum yield of C1-INH and a minimum yield of ACT.

The present invention has been described above by making reference to specific examples. These examples are by no means intended to restrict the present invention, but to illustrate the way in which the present invention works.

1. Process for the depletion of 1-antichymotrypsin (ACT) from a C1-INH preparation obtained from blood plasma by means of a preceding process involving several steps including, but not limited to hydrophobic interaction chromatography, wherein the depletion of ACT from the C1-INH preparation is carried out by anionic exchange chromatography and comprising the following steps:
(i) loading an anionic exchange chromatography column comprising a stationary phase with the C1-INH preparation under first conditions under which C1-INH and ACT bind to the stationary phase;
(ii) an optional step of washing the charged column;
(iii) application of second conditions so as to elute ACT by means of a mobile phase;
(iv) application of third conditions so as to elute C1-INH by means of a mobile phase,
characterized in that
the second condition consists in the use of an elution buffer of an ionic strength A and
the third condition consists in the use of an elution buffer of an ionic strength B, wherein ionic strengths A and B are different,
and wherein transition from ionic strength A to ionic strength B is achieved by means of a salt concentration gradient, or by means of a step elution using elution buffers EB_{A} and EB_{B} with different salt concentrations c_{A} and c_{B} and
wherein
(i) elution buffer EB_{A} has a conductivity of 18.7 to 20.2 mS/cm at 25° C., preferably of 18.9 to 19.8 mS/cm at 25° C., most preferably of 19.2 mS/cm at 25° C.
(ii) elution buffer EB_{B} has a conductivity being higher than 21.6 mS/cm at 25° C. wherein elution buffer EB_{B} is eluting C1-INH still bound to the anionic exchange chromatography column after elution step (i).

2. Process according to one or more of the preceding claims, wherein
(i) elution buffer EB_{A} consists of 10 mM Tris, 175-190 mM NaCl, preferably 175-185 mM NaCl, most preferably 180 mM NaCl, pH 7.2, and
(ii) elution buffer EB_{B} has a conductivity being higher than 25.3 mS/cm at 25° C. wherein elution buffer EB_{B} is eluting C1-INH still bound to the anionic exchange chromatography column after elution step (i).

3. Process according to one or more of the preceding claims, wherein
(i) elution buffer EB_{A} consists of 10 mM Tris, 175-190 mM NaCl, preferably 175-185 mM NaCl, most preferably 180 mM NaCl, pH 7.2, and
(ii) elution buffer EB_{B} consists of 10 mM Tris, 1M NaCl, pH 7.2

4. Process according to one or more of the preceding claims, wherein the stationary phase material belongs to the type of weak anion exchangers, such as Capto® DEAE (sold by GE, using diaminoethyl as a functional group) or preferably of strong anion exchangers, such as Q HP resin, Capto® Q Impres resin, Capto® Q resin (all sold by GE, all with quaternary ammonium as a functional group) or Fractogel® TMAE, Eshmuno® H (sold by Merck, with trimethylamonethyl as a functional group).

5. Process according to any one of the preceding claims, wherein the C1-INH preparation is derived from human blood plasma.

6. Process according to any one of the preceding claims, wherein the C1-INH preparation consists essentially of C1-INH and ACT dissolved in a medium.

7. C1-INH preparation that can be obtained by using a process according to any one of the preceding claims.
